(19) United States
(12) Patent Application Publication
(10) Pub. No.: US 2013/0119579 A1
(43) Pub. Date: May 16, 2013

(54) METHOD AND SYSTEM FOR PRODUCING AT LEAST ONE PATIENT-SPECIFIC SURGICAL AID

(71) Applicants: Joseph P. Iannotti, Strongsville, OH (US); Wael K. Barsoum, Bay Village, OH (US); Jason Bryan, Avon Lake, OH (US)

(72) Inventors: Joseph P. Iannotti, Strongsville, OH (US); Wael K. Barsoum, Bay Village, OH (US); Jason Bryan, Avon Lake, OH (US)

(73) Assignee: THE CLEVELAND CLINIC FOUNDATION, Cleveland, OH (US)

(21) Appl. No.: 13/622,460

(22) Filed: Sep. 19, 2012

Related U.S. Application Data
(60) Provisional application No. 61/536,756, filed on Sep. 20, 2011.

(51) Int. Cl. A61B 17/15

(52) U.S. Cl. CPC A61B 17/15
USPC 264/259; 264/334; 249/187.1

(57) ABSTRACT

A method for producing at least one patient-specific surgical aid includes providing a physical model of a native patient tissue. The physical model has at least one surface of interest. A constraining wall is placed in contact with at least a portion of the physical model. A moldable substance is placed into contact with at least a portion of the surface of interest. An impression of the surface of interest is maintained upon the moldable substance. The moldable substance is solidified into a patient-specific surgical aid. The patient-specific surgical aid is removed from the physical model. A system for producing at least one patient-specific surgical aid is also disclosed.

### RELATED APPLICATION

This application claims priority from U.S. Provisional Application No. 61/536,756, filed 20 Sep. 2011, the subject matter of which is incorporated herein by reference in its entirety.

### TECHNICAL FIELD

The present invention relates to an apparatus and method for producing at least one patient-specific surgical aid and, more particularly, to a method and apparatus for using a physical model of a native patient tissue to help produce at least one patient-specific surgical aid.

### BACKGROUND OF THE INVENTION

In the installation of a prosthetic shoulder joint into a patient's body, a glenoid component is implanted into the glenoid vault of the patient's scapula. An obverse surface of the glenoid component is configured for articulating contact with a humeral component carried by the patient's humerus. A reverse surface of the glenoid component is secured to the bone surface of the glenoid vault.

Because the shoulder prosthesis is normally provided to correct a congenital or acquired defect of the native shoulder joint, the glenoid vault or joint surface often exhibits a pathologic, nonstandard anatomic configuration. A surgeon must compensate for such pathologic glenoid vault anatomy when implanting the glenoid component in striving to achieve a solid anchoring of the glenoid component into the glenoid vault. Detailed preoperative planning, using two- or three-dimensional internal images of the shoulder joint, often assists the surgeon in compensating for the patient's anatomical limitations. During the surgery, an elongated pin may be inserted into the surface of the patient's bone, at a predetermined trajectory and location, to act as a passive landmark or active guiding structure in carrying out the preoperatively planned implantation. This “guide pin” may remain as a portion of the implanted prosthetic joint or may be removed before the surgery is concluded. This type of pin-guided installation may be useful in any joint replacement procedure—indeed, in any type of surgical procedure in which a surgeon-placed fixed landmark is desirable.

In addition, and again in any type of surgical procedure, modern minimally invasive surgical techniques may dictate that only a small portion of the bone or other tissue surface being operated upon is visible to the surgeon. Depending upon the patient's particular anatomy, the surgeon may not be able to precisely determine the location of the exposed area relative to the remaining, obscured portions of the bone through mere visual observation. Again, a guide pin may be temporarily or permanently placed into the exposed bone surface to help orient the surgeon and thereby enhance the accuracy and efficiency of the surgical procedure.

A carefully placed guide pin or other landmark, regardless of the reason provided, will reduce the need for intraoperative imaging in most surgical procedures and should result in decreased operative time and increased positional accuracy, all of which are desirable in striving toward a positive patient outcome. Co-pending U.S. patent application Ser. No. 13/282,509, filed 27 Oct. 2011 and titled “System and Method for Association of a Guiding Aid with a Patient Tissue” (the entire contents of which are incorporated herein by reference) discloses a guide, which may be patient-specific, for helping associate a landmark with a patient tissue. However, the guide of this co-pending application is described, in relevant part, as being planned using a computer and generated (e.g., via three-dimensional printing or rapid prototyping) with the landmark-guiding features in place. In contrast, a user may wish to manufacture or generate a guide without the use of a computer in some situations.

### SUMMARY OF THE INVENTION

In an embodiment of the present invention, a method for producing at least one patient-specific surgical aid is described. A physical model of a native patient tissue is provided. The physical model has at least one surface of interest. A constraining wall is placed in contact with at least a portion of the physical model. A moldable substance is placed into contact with at least a portion of the surface of interest. An impression of the surface of interest is maintained upon the moldable substance. The moldable substance is solidified into a patient-specific surgical aid. The patient-specific surgical aid is removed from the physical model.

In an embodiment of the present invention, a system of providing at least one patient-specific surgical aid is provided. A physical model of a native patient tissue is provided. The physical model has at least one surface of interest. A constraining wall for contacting at least a portion of the physical model is provided. A moldable substance for contacting at least a portion of the surface of interest and for maintaining an impression of the surface of interest thereupon is provided. The moldable substance is solidified into a patient-specific surgical aid. The patient-specific surgical aid is removed from the physical model for use.

### BRIEF DESCRIPTION OF THE DRAWINGS

For a better understanding of the invention, reference may be made to the accompanying drawings, in which:

FIGS. 1-3 are perspective views of one structure of the present invention in a first configuration;

FIGS. 4-6 are perspective views of the structure of FIGS. 1-3 in a second configuration;

FIGS. 7 and 9-10 are perspective views of the structure of FIGS. 1-3 in a third configuration;

FIG. 8A is a partial cross-sectional view taken along line A-A in FIG. 7;

FIG. 8B is a partial cross-sectional view taken along line B-B in FIG. 7;

FIG. 8C is a partial cross-sectional view taken along line C-C in FIG. 7;

FIGS. 11-13 are perspective views of another structure of the present invention; FIGS. 14A-14F are perspective views depicting an example sequence of operation of the present invention;

FIG. 15 is a perspective view of another structure of the invention in a first configuration;

FIGS. 16A-16C are partial cross-sectional views of the structure of FIG. 15; and

FIG. 17 is a partial cross-sectional view of another structure of the invention.

### DESCRIPTION OF EMBODIMENTS

The patient tissue is shown and described herein at least as a scapula and an acetabulum, and the prosthetic implant component is shown and described herein at least as a glenoid prosthetic shoulder component and an acetabular prosthetic hip component, but the patient tissue and corresponding prosthetic implant component could be any desired types such as, but not limited to, hip joints, shoulder joints, knee joints, ankle joints, phalangeal joints, metatarsal joints, spinal structures, long bones (e.g., fracture sites), or any other suitable patient tissue use environment for the present invention. The below description presumes that the system, apparatus, and method described is being used in conjunction with a surgical procedure (namely, an at-least-partial joint replacement or resurfacing), but the system, apparatus, and method described may be used in any desired manner and for any desired purpose without harm to the present invention.

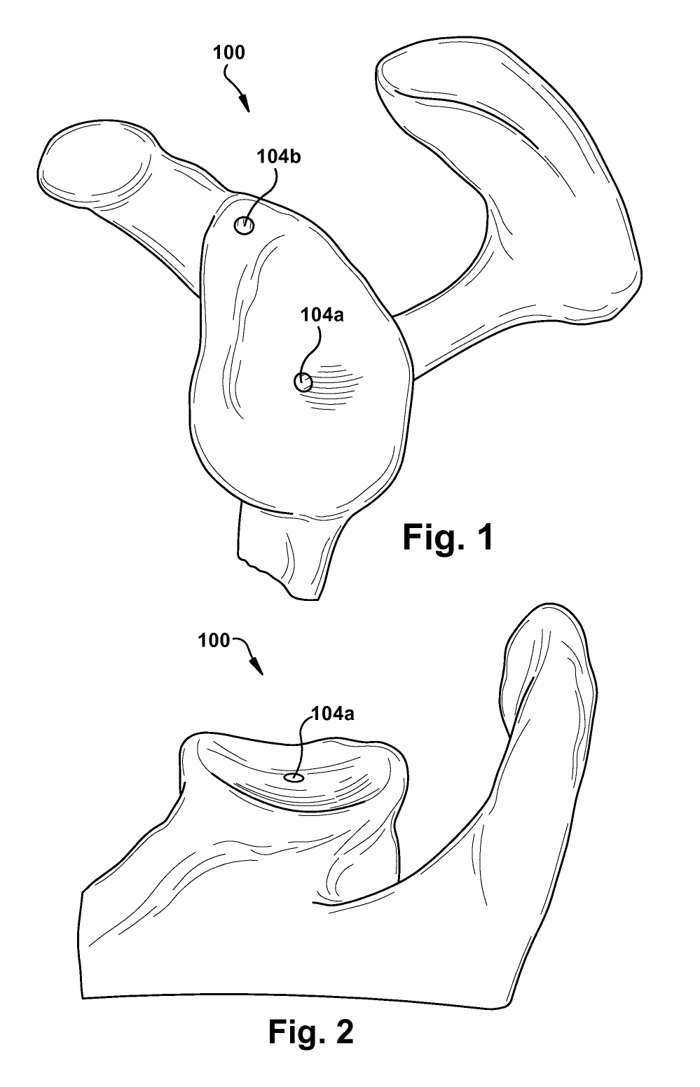

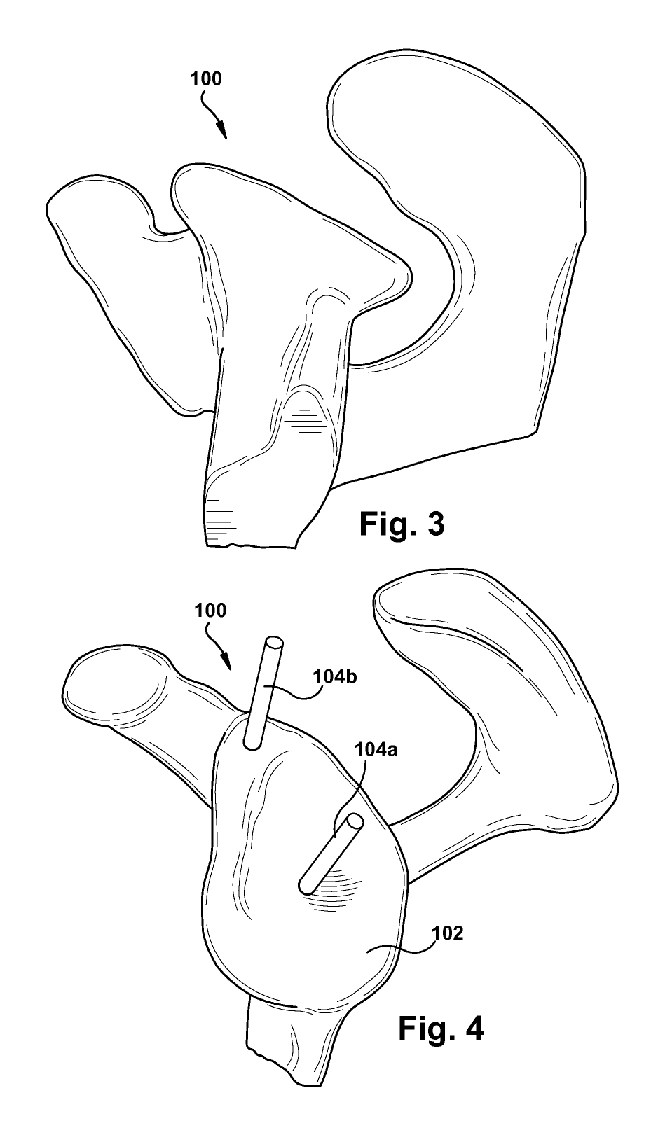

In accordance with the present invention, FIGS. 1-3 depict three different perspective views of a physical model 100 (sometimes called a “surrogate model”) of a native patient tissue—here, the glenoid fossa and surrounding scapular structures. The term “native patient tissue” and variants thereof is used herein to indicate a patient tissue of interest in its condition at the time of surgical preparation, having any included natural or artificial structures of interest, whether congenital or acquired. The term “model” is used herein to indicate a replica or copy of a physical item, at any relative scale and represented in any medium, physical or virtual. (However, herein the model will be presumed to be a physical model 100, as opposed to a virtual model located only on a computer system.) The patient tissue model may be a total or partial model of a subject patient tissue, and may be created in any suitable manner. For example, and as presumed in the below description, the patient tissue model may be a tangible representation of a virtual model generated using computer tomography (“CT”) data imported into a computer aided drafting (“CAD”) system. Additionally or alternatively, the patient tissue model may be based upon a virtual model created with the aid of digital or analog radiography, magnetic resonance imaging, or any other suitable imaging means. The patient tissue model will generally be displayed for the user to review and manipulate preoperatively, such as through the use of a physical model or (in the case of a virtual model) the use of a computer or other graphical workstation interface.

The patient's name, identification number, surgeon's name, and/or any other desired identifier may be molded into, printed on, attached to, or otherwise associated with the physical model 100 in a legible manner. Particularly when based upon a virtual model, the physical model 100 may be made by any suitable method such as, but not limited to, selective laser sintering (“SLS”), fused deposition modeling (“FDM”), stereolithography (“SLA”), laminated object manufacturing (“LOM”), electron beam melting (“EBM”), 3-dimensional printing (“3DP”), contour milling, computer numeric control (“CNC”), other rapid prototyping methods, or any other desired manufacturing process.

As examples of physical model 100 generation means omitting the step of the preoperative-imaging based virtual model, the physical model may be directly generated from the native patient tissue using a microscribe three-dimensional scanning/replicating device and/or using a molding system to take an impression of the patient's tissue from which the physical model can be made.

Regardless of how the physical model 100 comes into existence, it represents a three-dimensional, physically manipulable representation of a particular native patient tissue. The physical model 100 has at least one surface of interest 102 (substantially the glenoid fossa, in the embodiment shown in the Figures). The term “surface of interest” is used herein to indicate a surface of the physical model 100 which the user wishes to replicate and/or reference with the patient-specific surgical aid. As one of ordinary skill in the art will be aware, a “surface of interest” 102 in most cases will not have clearly defined borders, but that person of ordinary skill in the art will be able to instinctively differentiate between a surface of interest and another patient tissue, which is not a surface of interest, for a particular application of the present invention.

As an example, the physical model 100 of the Figures depicts a portion of a scapula which will be undergoing a glenoid resurfacing and/or replacement procedure. Therefore, one surface of interest 102 that will be referenced herein is the glenoid fossa surface. As shown in FIGS. 1-3, at least one landmark 104 may be associated with the physical model 100. Here, two landmarks 104a and 104b (each of which is a bore or aperture) are present, with the former being located on the glenoid fossa surface and the latter being located to one side of the glenoid fossa surface, shown here on the glenoid rim at/near the base of the coracoid process. The term “landmark” 104 is used herein to indicate any guiding aid which serves as a detectable indicator of a particular position on a “marked” substrate (here, the patient tissue or the physical model 100 representing such). The landmarks 104 discussed with respect to the present invention are presumed to be affixed or otherwise rigidly associated with a particular patient tissue so that a user can confidently maintain a sense of physical and/or visual orientation within the operative field. Suitable landmarks 104 may include, but are not limited to, visual “written” marks (e.g., a thin layer of a substance left behind after contact with a crayon, surgical pen, or the like), other written marks outside the visual spectrum (e.g., a UV-fluorescent paint), guide pins, fasteners (e.g., screws, nails, staples, or the like), radioactive tags, bovie cautery burn marks, metallic or nonmetallic devices attached to the desired landmark site (e.g., a rivet, tack, or the like), or even modifications of the patient tissue itself (e.g., notches, inscribed lines, drill holes, or the like, as with the landmarks 104a and 104b shown in the Figures).

The marking location and marking trajectory/orientation, as appropriate, of each landmark 104 on the physical model 100 may be predetermined by a user before the landmark is associated with the physical model. This predetermination may occur intraoperatively, while the user is able to directly see the condition of the surgical site and associate the landmark(s) 104 with the corresponding physical model 104 accordingly. However, it is also contemplated that a predetermination of the desired marking location and desired marking trajectory for each landmark 104 could be accomplished preoperatively, with reference to preoperative imaging of the patient tissue. For example, a system similar to that of co-pending U.S. patent application Ser. No. 13/282,550, filed 27 Oct. 2011 and titled “System of Preoperative Planning and Provision of Patient-Specific Surgical Aids”, the entire contents of which are incorporated herein by reference, or any suitable preoperative planning system could be used. Using this or any other planning means (including “dead reckoning”, “eyeballing”, or other non-planned or non-assisted placement methods), a user can create a physical model 100 for observation, manipulation, rehearsal, or any other pre-operative tasks, having any number and type of landmarks 104 associated therewith, for any reason(s).

Optionally, and particularly when a computer-assisted pre-operative planning method is used, virtual landmarks 104 may be virtually placed on a virtual patient tissue model. In order to transfer those virtual landmarks 104 to the physical world for intra-operative use, the physical model 100 may be at least partially custom-manufactured responsive to preoperative imaging of the patient tissue, the physical model 100 having at least one landmark 104 associated therewith as generated.

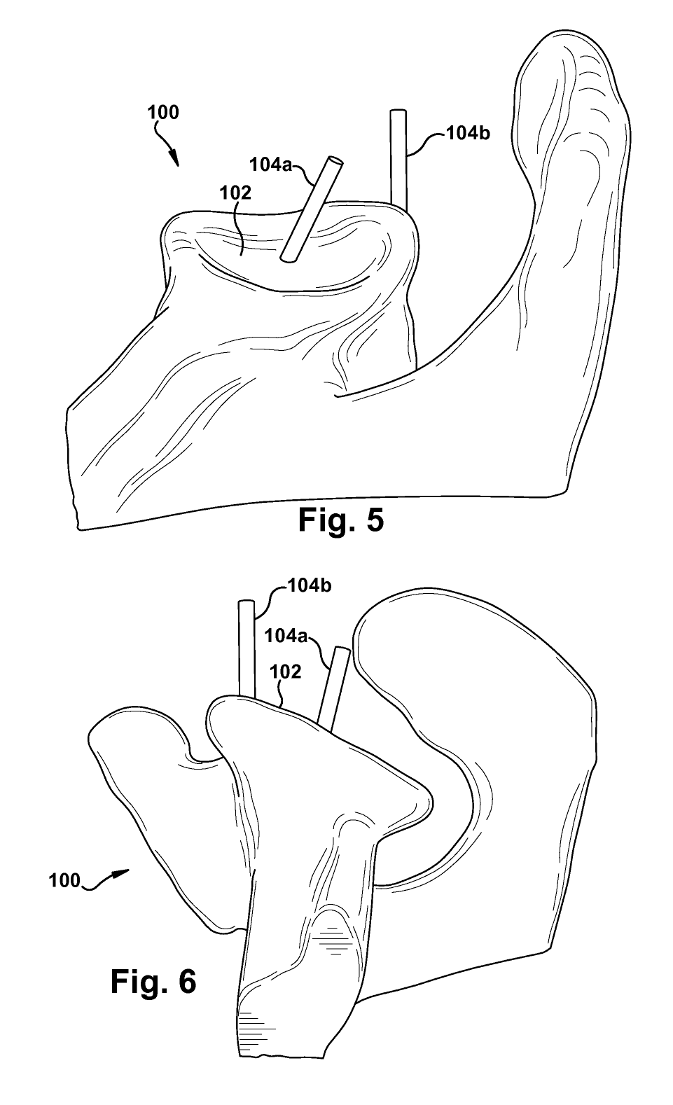

Turning to FIGS. 4-6, three perspective views are shown of a physical model 100 having landmarks 104a, 104b which are guide pins, protruding from the surface of the physical model. The guide pin landmarks 104a, 104b could either be stock or bespoke guide pins inserted into previously provided bores in the physical model 100, or could be originally formed as protrusions from the physical model. Unlike two-dimensional “marking” landmarks which merely denote a marking location, the guide pin landmarks 104a, 104b shown in FIGS. 4-6 are three-dimensional and therefore include both a marking location and a marking trajectory.

Speaking more generally, the guide pin landmarks 104a, 104b of FIGS. 4-6 are examples of non-native (i.e., non-native patient tissue) structures which are associated with the surface of interest 102 (For certain use environments of the present invention, the glenoid rim guide pin landmark 104b may be considered to be “outside” the surface of interest 102, depending on the metes and bounds of the surgical procedure being planned.). Optionally, and as is the case with the guide pin landmarks 104a, 104b of FIGS. 4-6, the non-native structure may be an information feature providing clinically useful information to a user.

“Clinically useful” information is used herein to indicate any information, other than the structure of the native patient tissue itself, that assists one of ordinary skill in the art with some pre- and/or intra-operative task. An “information feature” is any physical feature or characteristic of the physical model 100 which signifies or communicates the clinically useful information to the user, optionally in combination with a preoperative plan. Optionally, the information feature may be substantially separated from the surface of interest.

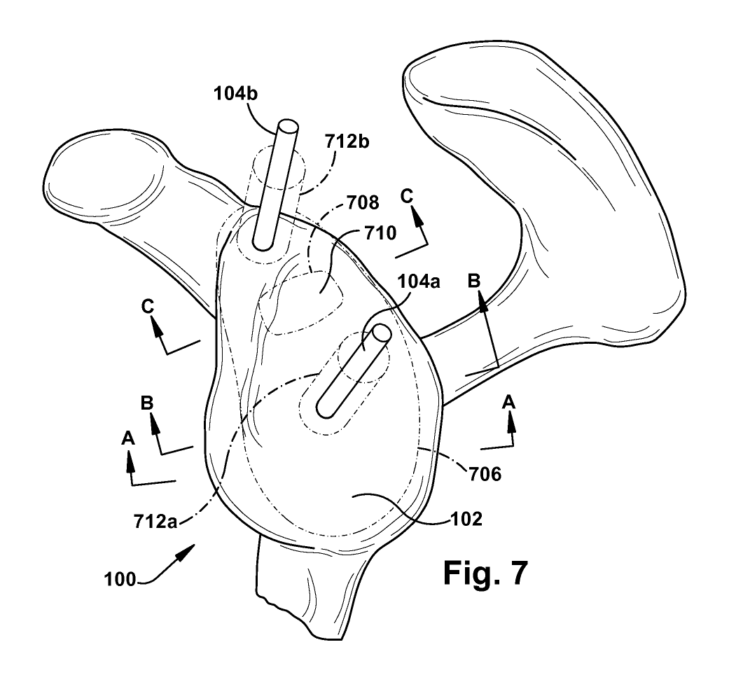

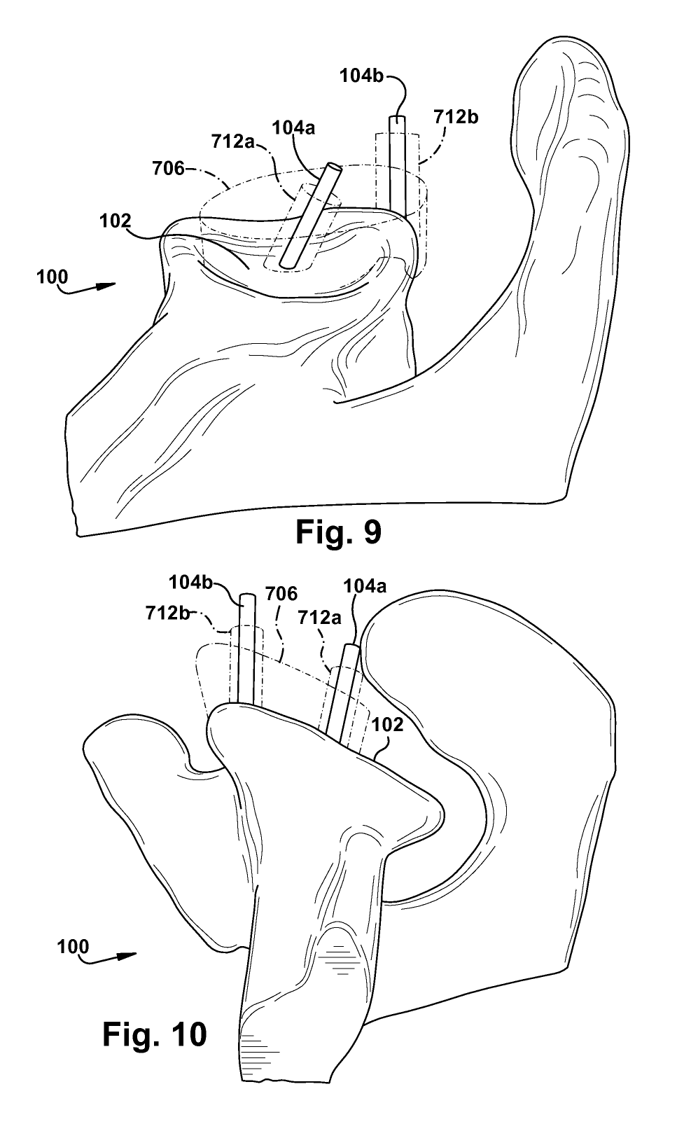

FIGS. 7-10 illustrate various structures which can assist with providing at least one patient-specific surgical aid using a molding fabrication process according to the present invention. FIGS. 7-10 each include at least a portion of a physical model 100 having two guide pin landmarks 104a, 104b protruding therefrom. FIGS. 7-10 each also depict at least one constraining wall which may be provided for contacting at least a portion of the physical model 100, such as at least a portion of the surface of interest 102. For example, and as shown in FIG. 7, four of these optional constraining walls are shown schematically via dotted lines: an outer wall 706 defining at least one perimeter boundary of a patient-specific surgical aid, an inner wall 708 defining at least one inner recess 710 of the patient-specific surgical aid (omitted from FIGS. 9-10 for clarity), and two guide bushings 712a and 712b which each are associated with a guide pin landmark 104a and 104b, respectively, to define a guiding aperture (not shown) through a thickness of the patient-specific surgical aid, as will be discussed below.

The constraining wall(s) of the present invention, when provided, may be of any suitable size, shape, configuration, material, construction, or other physical property, and may be integrally formed in a one-piece manner with the physical model 100 or separately provided by any agent, at any time, and with any degree (or none) of attachment or connection, permanent or temporary, to the physical model 100. The dimensions, construction, material(s), configuration(s), attachment(s), and other properties of a suitable constraining wall(s) may be readily determined by one of ordinary skill in the art for a particular application of the present invention. Because of the wide range of possible arrangements and constructions available for such, the constraining walls are simply shown schematically as dotted lines in FIGS. 7 and 9-10.

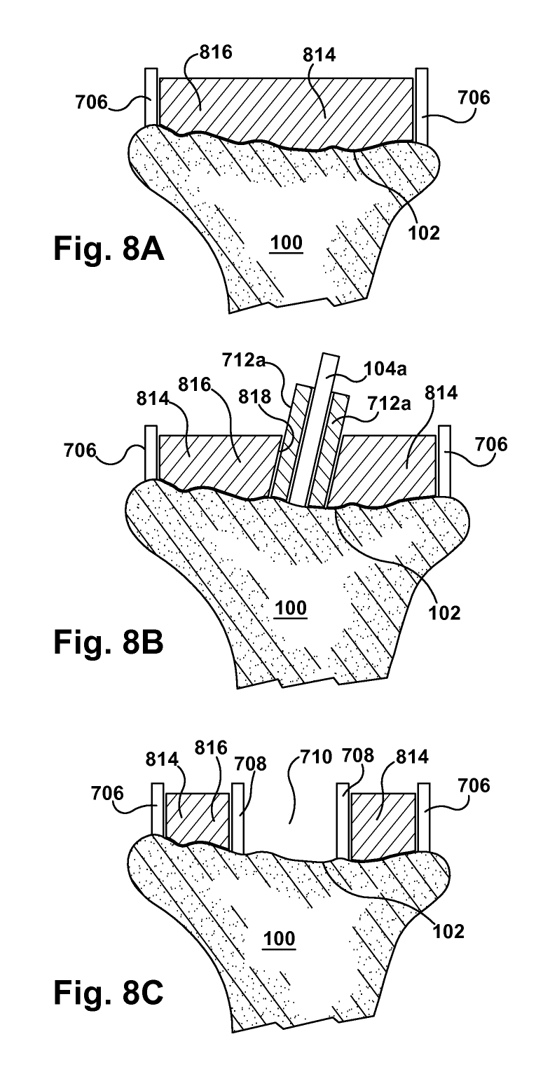

Regardless of their specific properties, each constraining wall is contemplated for use in helping to form a patient-specific surgical guide 814, as shown in the cross-sectional views of FIGS. 8A, 8B, and 8C. The patient-specific surgical guide 814 will, for most applications of the present invention, be molded from the physical model 100, optionally with the assistance of one or more constraining walls. That is, a moldable substance 816 will be placed into contact with at least a portion of the surface of interest 102. (In FIGS. 8A-8C, the moldable substance 816, which makes up the body of the patient-specific surgical guide 814 as denoted by the shaded portions of those Figures, is shown as being slightly separated from adjacent surfaces for clarity of depiction. In reality, the moldable substance 816 can be placed into a mating relationship with those adjacent surfaces, as desired by a user.)

The moldable substance 816 may be any suitable material, or combination of materials, which is capable of maintaining an impression of the surface of interest 102 thereupon. Examples of suitable reusable or single-use moldable substances include, but are not limited to, modeling clay, gelatins, urethane and silicone rubber, urethane and epoxy casting resins, other epoxies, latexes, adhesives, cements (e.g., bone cement or any other type), glues, foams (e.g., florists' foam, aerosol foams, or any other type), other plastic materials, candy, closely packed wadding/gauze/batting, powders, putty, pinscreen-based devices (e.g., structures using similar principles to those disclosed in U.S. Pat. No. 4,654,989, issued Apr. 7, 1987 to Ward Fleming), and the like. Particularly if the moldable substance 816 has relatively high viscosity and/or is a solid, one or more of the constraining walls discussed herein may be omitted if not needed to constrain a moldable substance that itself has sufficient physical properties to maintain position as desired by the user.

The moldable substance 816 should be able to be solidified into a patient-specific surgical aid 814, which can then be removed from the physical model 100 for use. The term “solidify” is used herein to indicate that the moldable substance 816 dries, sets, cures, or otherwise takes on a definite physical form (optionally with the use of an oven, fan, light of a certain wavelength [e.g., ultraviolet], or other “curing” aid) sufficient to substantially maintain the impression of the surface of interest 102 upon removal of the patient-specific surgical aid 814 from the physical model 100. “Solidify” is also used herein to reference the process, if any, of “finalizing” a configuration of a moldable substance 816 that is substantially solid in raw material form (e.g., florists' foam) sufficiently for the moldable substance to maintain the format of the patient-specific surgical aid 814, whether or not any phase change from raw to “solidified” form of the moldable substance occurs. A “solidified” patient-specific surgical aid 814 may still be somewhat pliant or supple, or even include portions (e.g., “pockets”) of fluid material, and be considered sufficiently “solidified” for a particular application. Conversely, the “solidified” patient-specific surgical aid 814 may be substantially rigid.

Optionally, a release agent or other intermediate substance (e.g., a lubricant, mold release spray or powder, wax, thin film [e.g., plastic wrap], or the like) may be placed on at least a portion of the surface of interest 102 before the moldable substance 816 is placed into contact with at least a portion of the surface of interest, for any desired reason including protecting the surface of interest from the moldable substance or vice versa, facilitating removal of the patient-specific surgical aid 814 from the physical model 100, or for any other reason. In this case, the contact between the moldable substance 816 and the affected portion(s) of the surface of interest 102 may be indirect.

Each constraining wall (e.g., the outer wall 706, inner wall 708, guide bushing 712, or any other constraining wall as desired) may be provided and used to help contain or block flow of the moldable substance 816 before it is solidified, to help define the area of the physical model 100 which is a surface of interest 102, to help insulate or separate portions of the patient-specific surgical aid 814 from each other, and/or for any other desired reason. The moldable substance 816 may come into contact with at least a portion of the constraining wall—this is evident in FIGS. 8A-8C, in which a relatively fluid moldable substance has been poured into a mold cavity comprised of the surface of interest 102 and the constraining walls. To facilitate the molding process, the physical model 100 may be arranged with a substantial part of the surface of interest 102 being located at a topmost position (if it is not already), and optionally oriented to be substantially level to a local ground plane, before the moldable substance 816 is brought into contact with the surface of interest—in this manner, a suitable amount of the moldable substance may be assisted by gravity in reaching all desired portions of the surface of interest. However, it is contemplated that the constraining wall(s) need not solely provide containment of the moldable substance 816, and other structures (not shown) might also be used to facilitate the molding process. Additionally or alternatively, it is contemplated that a portion of the moldable substance 816 may be molded without need for containment, due to the viscosity of the moldable substance 816, the orientation of the physical model 100 with respect to the local gravitational field, or for any other reason.

Optionally, at least one constraining wall may be incorporated into, and become a part of, the patient-specific surgical aid 814 during and/or via the molding process (for example, to help give rigidity and structure to the patient-specific surgical aid). In such case, the affected constraining wall(s) may be detached or otherwise removed from contact with the physical model 100 as the patient-specific surgical aid 814 is removed from the physical model.

FIG. 8A depicts a simple cross-section through the thickness of the patient-specific surgical aid 814, wherein the moldable substance 816 has been poured into the outer wall 706. The outer wall 706 may be placed somewhat arbitrarily to provide an outer border for the patient-specific surgical aid 814. Alternately, at least a portion of the outer wall 706 itself may be an information feature and contain clinically useful information, such as a “cookie cutter” type border indicator showing where the underlying patient tissue should be resected.

FIG. 8B depicts a slightly more complex cross-section than in FIG. 8A, with at least one non-native structure (here, the guide pin landmark 104a) bring associated with the physical model 100 in this cross-section. Here, a molded feature incorporated in the patient-specific surgical aid 814 is based upon the guide pin landmark 104a. That is, a bore or aperture 818 protrudes through the patient-specific surgical aid 814 as a negative space where the guide pin landmark 104a has prevented the presence of the moldable substance 816. Optionally, and as shown here, the guide bushing 712 or at least a portion of another constraining wall may be interposed between at least a portion of the non-native structure and the moldable substance 816. When the patient-specific surgical aid 814 is solidified and removed from the physical model 100, the guide pin landmarks 104 may either go with, and be considered a part of, the patient-specific surgical aid or may be removed therefrom.

Particularly when the diameter of the guide pin is chosen to correspond to a desired drill bit size, the guide pin landmarks 104 may assist with creating a drill guide aperture 818 in the patient-specific surgical aid 814, the drill guide aperture having the desired marking location and marking trajectory embodied in the corresponding guide pin landmark. Because contact between a rotating tool (such as a drill bit) and the moldable substance 816 forming the body of the patient-specific surgical aid 814 may degrade or break down the walls of the drill guide aperture 818, a guide bushing 712 type constraining wall (particularly if made of an abrasion-resistant material, such as stainless steel) may become an integral part of the patient-specific surgical aid 814 to limit the size of the aperture, resist abrasion (by preventing contact between the drill bit and the moldable substance 816), and/or serve as a guide for the drill bit.

FIG. 8C depicts a cross-section in which the inner recess 710 has been left free of moldable substance 816 for some reason. For example, the user may want to maintain a direct line of sight with the surface of interest 102 in that area, the inner recess 710 may provide material cost and/or weight savings for the patient-specific surgical aid 814, the inner recess 710 may mark a desired cutting plane for a step in the surgical procedure, the inner recess may be configured to accept a handle or other auxiliary tool structure, a portion of the patient tissue within the inner recess may be pressure-sensitive, or the inner recess may have been provided for any other reason. When the patient-specific surgical aid 814 is removed from the physical model 100, the inner recess 710 will remain “open”, and the inner wall 708 may be incorporated into the patient-specific surgical aid to maintain integrity (e.g., size and/or shape) of the inner recess. However, the inner recess 710 is omitted from successive Figures for clarity of depiction.

FIGS. 9-10 illustrate the manner in which the constraining walls (i.e., the outer wall 706 and guide bushings 712a and 712b) relate to the physical model 100 before and during the molding process for the patient-specific surgical aid 814.

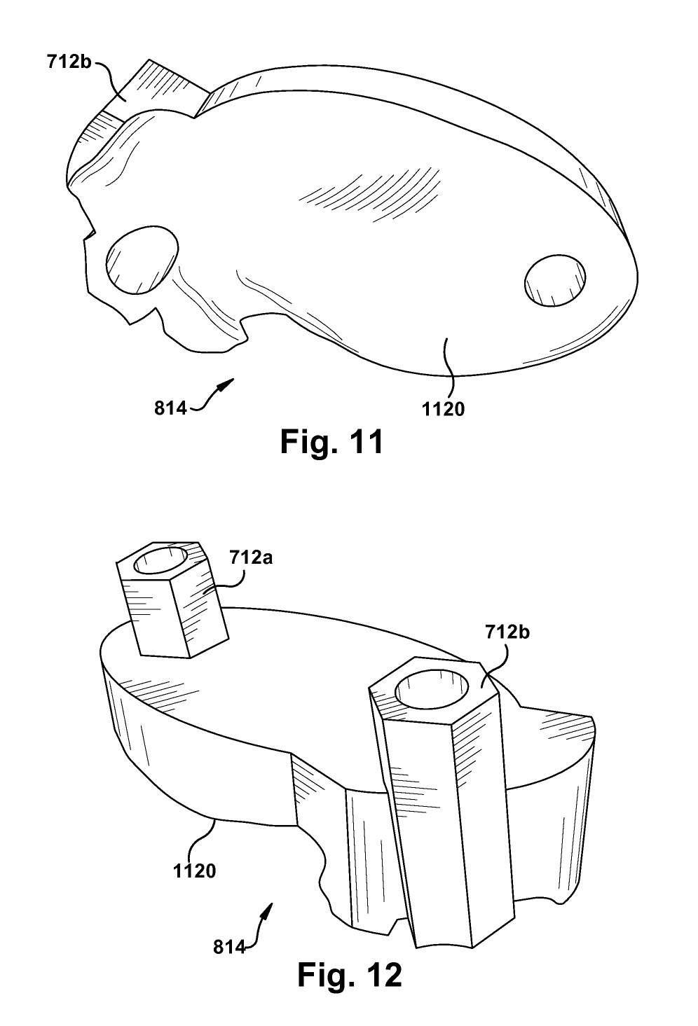

FIGS. 11-13 depict various perspective views of the completed patient-specific surgical aid 814 after having been solidified and removed from a physical model 100, such as the physical model 100 shown in FIGS. 9-10. As can be seen from the substantially bottom view of FIG. 11, the bottom surface 1120 substantially reproduces the contours of the surface of interest 102. Accordingly, the patient-specific surgical aid 814 produced using the above-described process should substantially mate with a native patient tissue (corresponding to the physical model 100) when placed upon the appropriate native patient tissue and aid the user in positively transferring landmark 104 marking locations and/or marking trajectories, or any other clinically useful information embodied in the patient-specific surgical aid to the native patient tissue. For example, the patient-specific surgical aid 814 may be used similarly to the devices disclosed in co-pending U.S. patent application Ser. No. 13/282,509, filed 27 Oct. 2011 and titled “System and Method for Association of a Guiding Aid with a Patient Tissue”, the entire contents of which are incorporated herein by reference.

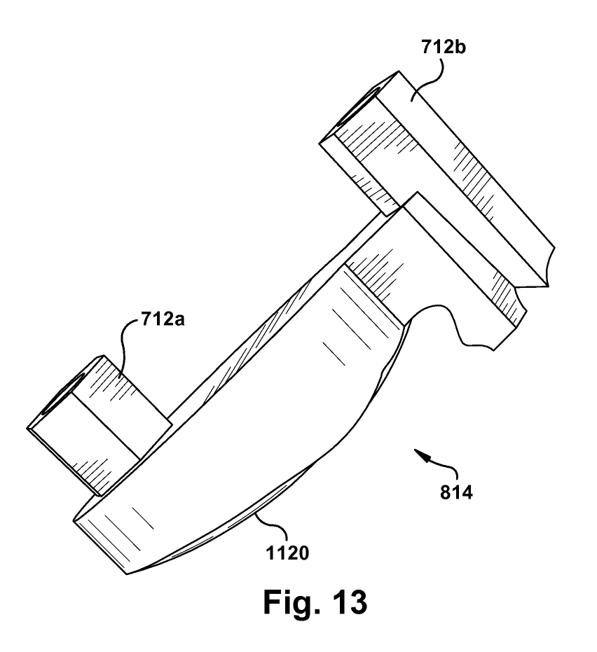

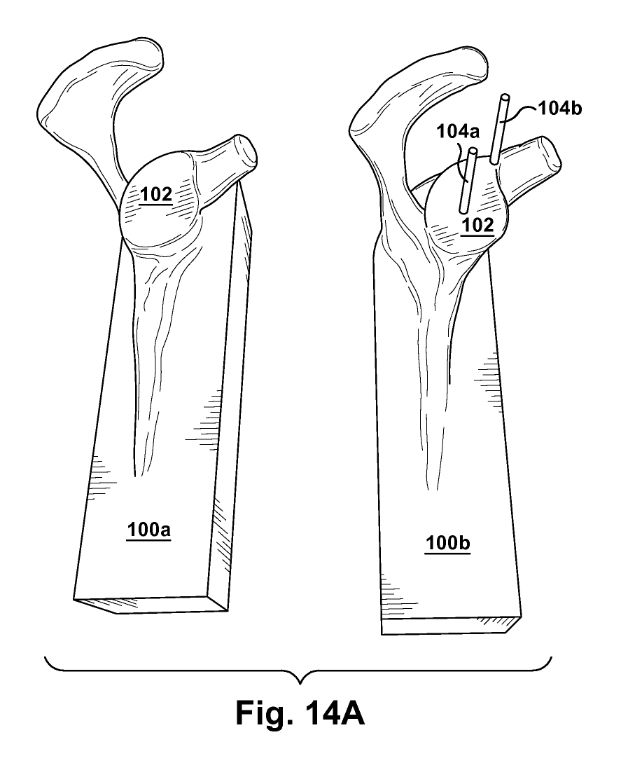

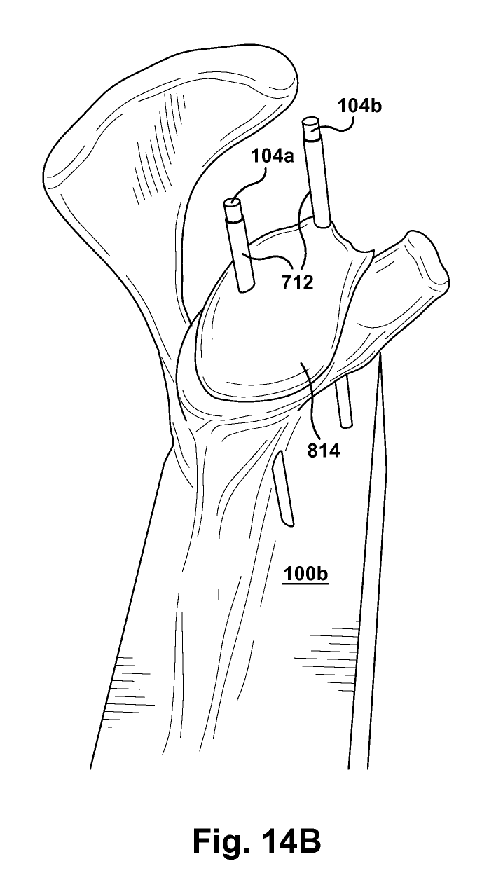

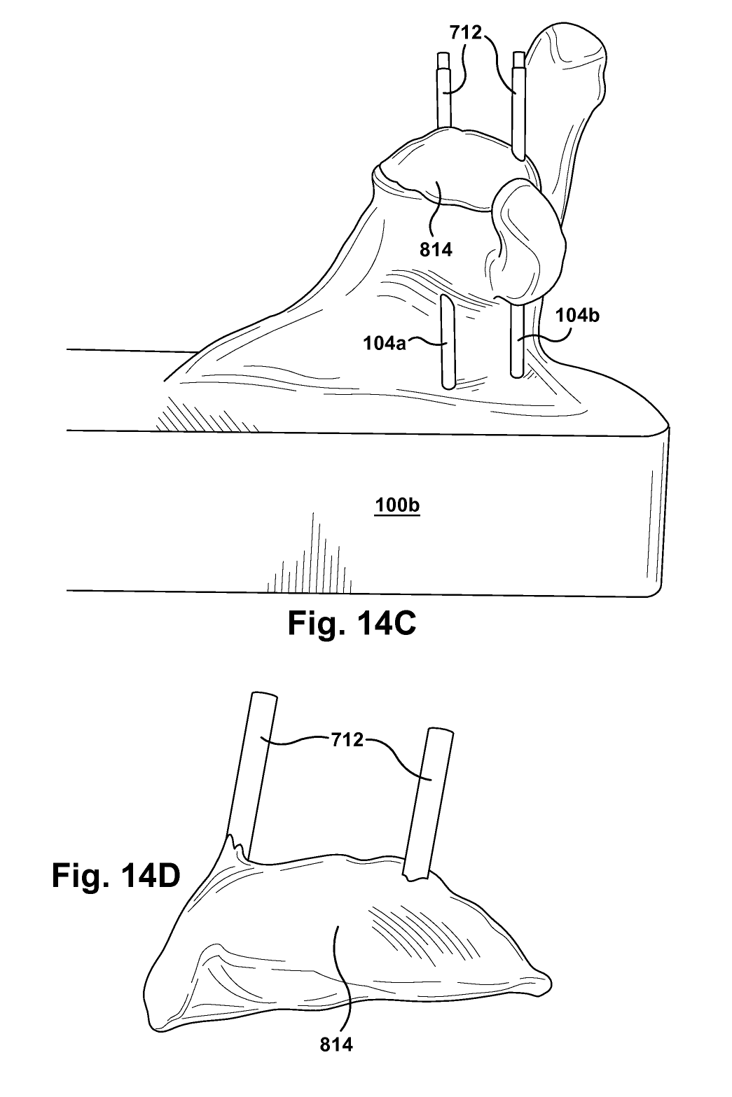

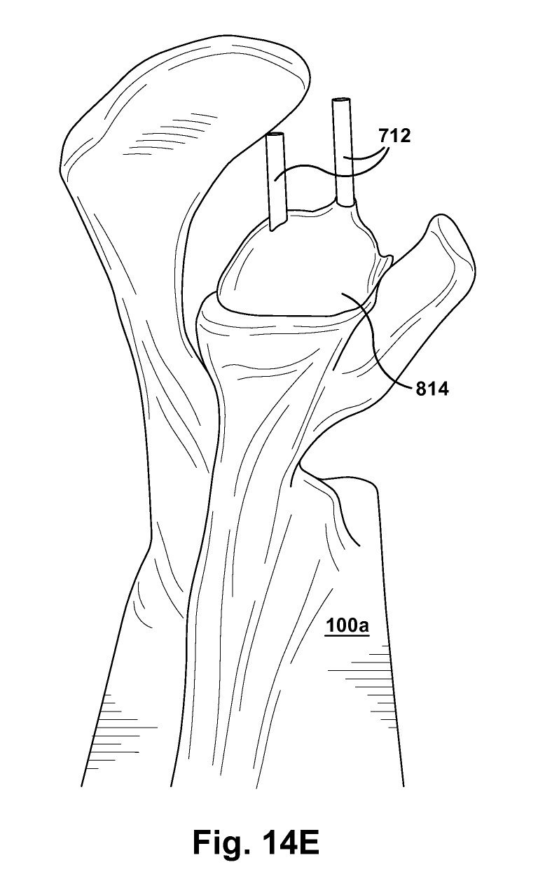

With reference to FIGS. 12-13, the guide bushings 712a, 712b have been incorporated into the patient-specific surgical aid 814, such as to guide a drill bit or guide pin into the underlying native patient tissue at a specified location and trajectory. Because the patient-specific surgical aid 814 is contemplated to only mate securely with the native patient tissue in a single relative orientation, the user has a reasonably high degree of confidence that the drill bit or guide pin is being guided into the underlying native patient tissue in the desired location and/or trajectory.

Accordingly, at least a part of the patient-specific surgical aid 814 is a patient-specific, single-use, bespoke component suited only for use at a surgical site corresponding to the surface of interest 102, though one of ordinary skill in the art could create a guide (not shown) which uses a patient-specific “disposable” structure (which may be substantially limited to the surface of interest) connected to a stock, generic “reusable” carrier (which may help the user in manipulating, stabilizing, securing, or otherwise interacting with the “disposable” structure as desired.

FIGS. 14A-14F depict an example sequence of operation of the present invention. FIG. 14A includes two physical models 100a (on the left, in the orientation of FIG. 14A) and 100b (on the right, in the orientation of FIG. 14A). Physical model 100a is bare, with no landmarks included. Physical model 100b includes landmarks 104a and 104b, which are each guide pins as previously described. In FIG. 14B, guide bushings 712, serving as constraining walls, have been placed around each of the landmarks 104a and 104b. FIG. 14C depicts the physical model 100b with the inserted landmarks 104a and 104b and the surrounding guide bushings 712. In FIG. 14C, a moldable substance 816 has been placed into contact with at least a portion of the surface of interest 102. Once the moldable substance 816 has solidified sufficiently to maintain an impression of the surface of interest 102 thereupon, it is removed and serves as a patient-specific surgical aid 814, seen alone in FIG. 14D. Here, the guide bushings 712 are incorporated into the patient-specific surgical aid 814. FIGS. 14E-14F depict different views of the patient-specific surgical aid 814 placed in a use position atop the bare physical model 100a, which includes no landmarks 104. (This bare physical model 100a is used in FIGS. 14E-14F as an easily viewed proxy for the patient tissue upon which that physical model was based, for the sake of the present discussion.) As can be seen in FIGS. 14E-14F, the guide bushings 712 are each held by the patient-specific surgical aid 814 at a location and trajectory that will direct a guide pin inserted therethrough into an insertion location and trajectory that substantially replicates the landmarks 104a and 104b present on the physical model 100b which was used to create the patient-specific surgical aid.

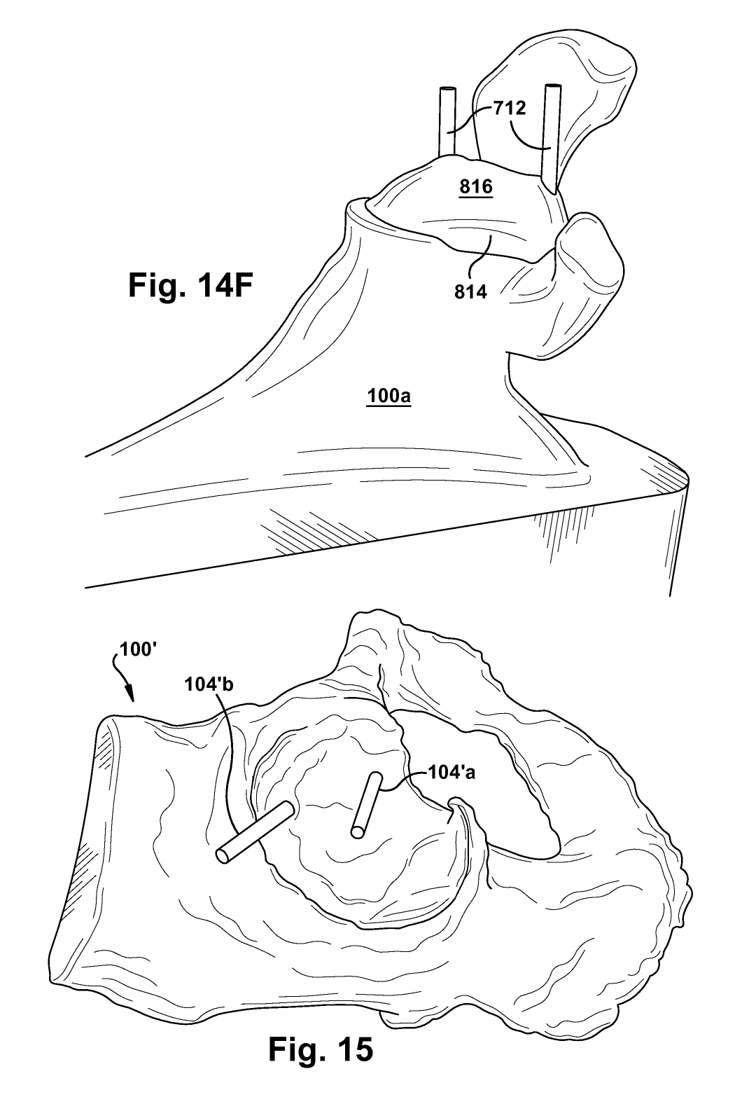

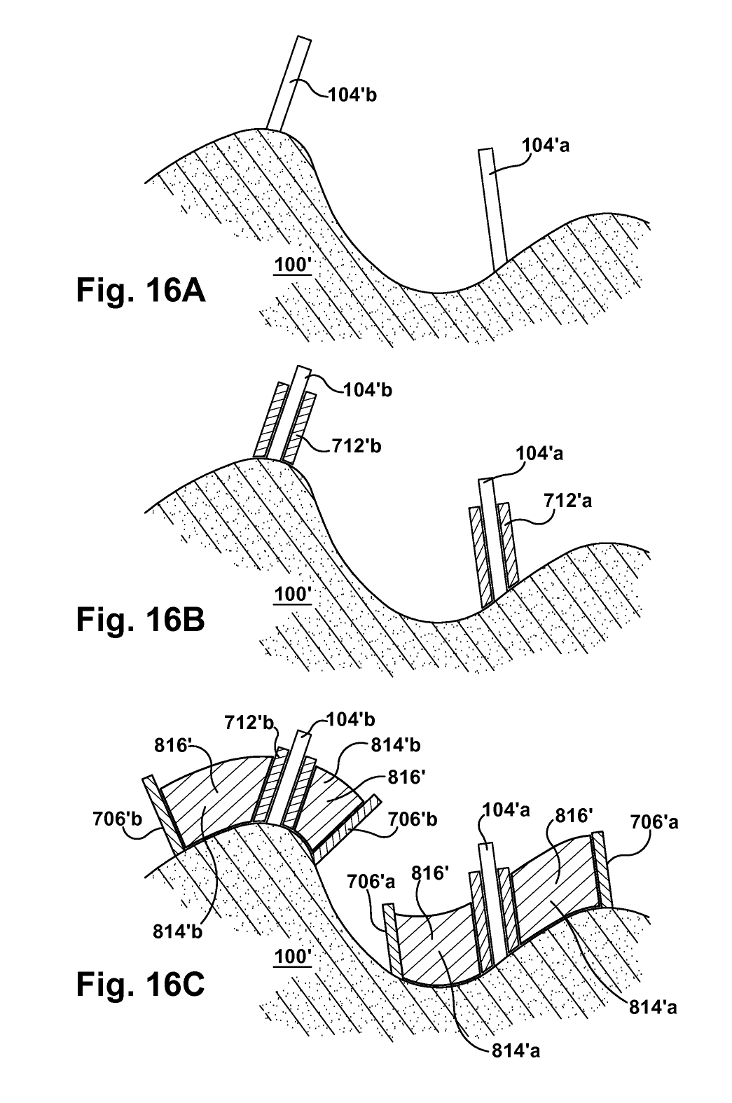

FIGS. 15-16C illustrate a second embodiment of the present invention for use with a second physical model 100′. The embodiment of FIGS. 15-16C is similar to the embodiment of FIGS. 1-14F and therefore, structures of FIGS. 15-16C that are the same as or similar to those described with reference to FIGS. 1-14F have the same reference numbers with the addition of a “prime” mark. Description of common elements and operation similar to those in the previously described embodiments will not be repeated with respect to the second embodiment.

As shown in FIG. 15, the physical model 100′ of the patient tissue embodies a hip and an acetabulum, as opposed to the scapula and glenoid of FIGS. 1-14F. In FIG. 15, two landmark 104′ guide pins have been placed, with one landmark 104′a being located inside the acetabulum and a second landmark 104′b being located outside the rim of the acetabulum.

Because the acetabulum is a relatively large void in a patient tissue, the user will probably want to avoid creating a single patient-specific surgical aid 814′ which includes both of the landmarks 104′a and 104′b in the positions depicted in FIG. 15, at least because it would take a relatively large quantity of moldable substance 816′ to fill the acetabulum and also come into molding contact with the area of the pelvis near the second landmark 104′b. That large quantity of moldable substance 816′ may be expensive, difficult to obtain, difficult to work with (e.g., does not solidify well), or may otherwise be undesirable to use.

Accordingly, while a single patient-specific surgical aid 814′ could be created in this situation, FIGS. 16A-16C depict, schematically and sequentially, the creation of two spaced-apart surgical aids 814′a and 814′b, which is contemplated as being a more likely scenario for most users of the present invention. FIG. 16A is a cross-sectional view of the physical model 100′ including both landmarks 104′a and 104′b. In FIG. 16B, guide bushings 712′a and 712′b have been placed around the landmarks 104′a and 104′b, respectively.

FIG. 16C shows the landmarks 104′a and 104′b, the guide bushings 712′a and 712′b, and two outer walls 706′a and 706′b, with each outer wall substantially surrounding one landmark-guide bushing pair. FIG. 16C also shows moldable substance 816′a and 816′b, which has been provided within the outer walls 706′a and 706′b, respectively. The moldable substance 816′a and 816′b may be the same at each location, or may differ, as desired by the user. For example, the moldable substance 816′b at/near the acetabular rim may be more viscous than the moldable substance 816′a within the acetabulat cavity, particularly if the orientation of landmark 104′b and/or outer wall 706′b would make the moldable substance 816′b prone to slide off the acetabular rim without achieving sufficient thickness to provide the desired bottom surface 1120 for the completed patient-specific surgical aid 814′a.

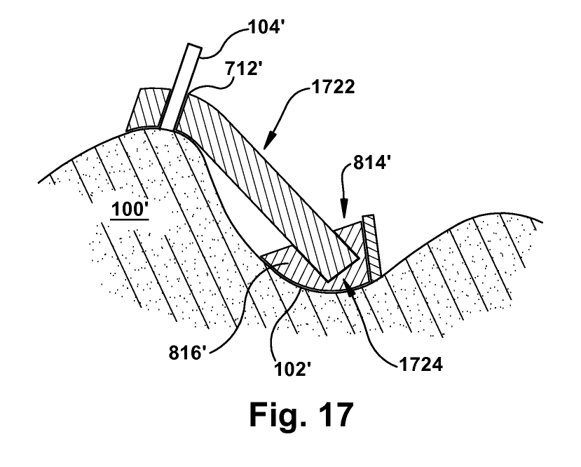

FIG. 17 illustrates an alternate patient-specific surgical aid 814′ in an example use environment corresponding to the second physical model 100′. As shown in FIG. 17, a landmark 104′ can be located at a position, such as the depicted acetabular rim, with a constraining wall having a guide bushing 712′ which is supported by a remote locator 1722. An anchor end 1724 of the remote locator 1722 is located adjacent, and optionally contacting, the physical model 100′ at a location which is provided with a moldable substance 816′. Here, the moldable substance 816′ is constrained by an outer wall 706′, to reduce the volume of moldable substance needed to replicate the surface of interest 102′ (here, the acetabular fovea) while securing the anchor end 1724 of the remote locator 1722 sufficiently to memorialize the location and/or trajectory of the landmark 104′ in the patient-specific surgical aid 814′ for transference to the native patient tissue.

The remote locator 1722, as well as any constraining wall feature supported thereby, may be at least partially patient-specific (e.g., designed and/or produced with the aid of pre-operative images of the native patient tissue), or may be a generic/stock component. The body of the remote locator 1722 could be configured to mate with, or follow closely along a contour of, a native patient tissue, or could instead have no particular relationship with the native patient tissue save that needed to span the distance between the landmark 104′ or other desired remote endpoint and the location at which the moldable substance 816′ is applied to create the patient-specific surgical aid 814′. While aspects of the present invention have been particularly shown and described with reference to the preferred embodiment above, it will be understood by those of ordinary skill in the art that various additional embodiments may be contemplated without departing from the spirit and scope of the present invention. For example, the specific methods described above for using the described system are merely illustrative; one of ordinary skill in the art could readily determine any number of tools, sequences of steps, or other means/options for virtually or actually placing the above-described apparatus, or components thereof, into positions substantially similar to those shown and described herein. Any of the described structures and components could be integrally formed as a single piece or made up of separate sub-components, with either of these formations involving any suitable stock or bespoke components and/or any suitable material or combinations of materials; however, the chosen material(s) should be biocompatible for most applications of the present invention. The mating relationships formed between the described structures need not keep the entirety of each of the “mating” surfaces in direct contact with each other but could include spacers or holdaways for partial direct contact, a liner or other intermediate member for indirect contact, or could even be approximated with intervening space remaining therebetween and no contact. Though certain components described herein are shown as having specific geometric shapes, all structures of the present invention may have any suitable shapes, sizes, configurations, relative relationships, cross-sectional areas, or any other physical characteristics as desirable for a particular application of the present invention. Any structures or features described with reference to one embodiment or configuration of the present invention could be provided, singly or in combination with other structures or features, to any other embodiment or configuration, as it would be impractical to describe each of the embodiments and configurations discussed herein as having all of the options discussed with respect to all of the other embodiments and configurations. Clinically useful information could include written or other legible information, as well as spatial or other physically discernible information. An air knife, water stream, or other fluid/dynamic barrier could be used as a constraining wall. The system is described herein as being used to plan and/or simulate a surgical procedure of implanting one or more prosthetic structures into a patient's body, but also or instead could be used to plan and/or simulate any surgical procedure, regardless of whether a non-native component is left in the patient's body after the procedure. A device or method incorporating any of these features should be understood to fall under the scope of the present invention as determined based upon the claims below and any equivalents thereof.

Other aspects, objects, and advantages of the present invention can be obtained from a study of the drawings, the disclosure, and the appended claims.

1. A method for producing at least one patient-specific surgical aid, the method comprising the steps of:
providing a physical model of a native patient tissue, the physical model having at least one area representing a patient tissue which is a surface of interest and at least one area representing a patient tissue which is not a surface of interest;
placing a constraining wall in contact with at least a portion of the physical model, the constraining wall separating at least a portion of an area of the physical model representing the patient tissue which is a surface of interest from at least a portion of an area of the physical model representing a patient tissue which is not a surface of interest;
placing a moldable substance into contact with at least a portion of the surface of interest;
maintaining an impression of the surface of interest upon the moldable substance;
solidifying the moldable substance into a patient-specific surgical aid; and
removing the patient-specific surgical aid from the physical model.

2. The method of claim 1, wherein the constraining wall is an outer wall and defines at least one perimeter boundary of the patient-specific surgical aid.

3. The method of claim 1, wherein the constraining wall is an inner wall and defines at least one inner recess of the patient-specific surgical aid.

4. The method of claim 3, wherein the constraining wall is a guide bushing and defines a guiding aperture through a thickness of the patient-specific surgical aid.

5. The method of claim 1, including the steps of:
associating at least one non-native structure with the surface of interest; and
incorporating in the patient-specific surgical aid a molded feature based upon the non-native structure.

6. The method of claim 5, wherein the non-native structure is an information feature providing clinically useful information to a user.

7. The method of claim 5, including the step of interposing at least a portion of the constraining wall between at least a portion of the non-native structure and the moldable substance.

8. The method of claim 1, including the step of incorporating the constraining wall into the patient-specific surgical aid.

9. The method of claim 1, including the step of placing the moldable substance into contact with at least a portion of the constraining wall.

10. The method of claim 1, including the step of placing a intermediate substance on at least a portion of the surface of interest before placing the moldable substance into contact with at least a portion of the surface of interest.

11. The method of claim 1, including the step of arranging the physical model with the surface of interest being substantially located at a topmost position before the step of placing a moldable substance into contact with at least a portion of the surface of interest.

12. The method of claim 1, wherein the step of providing a physical model of a native patient tissue includes the steps of:
creating a virtual model of a native patient tissue; and
creating a physical model of the native patient tissue as a tangible representation of the virtual model of the native patient tissue.

13. A system of providing at least one patient-specific surgical aid, the system comprising:
a physical model of a native patient tissue, the physical model having at least one area representing a patient tissue which is a surface of interest and at least one area representing a patient tissue which is not a surface of interest;
a constraining wall for contacting at least a portion of the physical model, the constraining wall separating at least a portion of an area of the physical model representing the patient tissue which is a surface of interest from at least a portion of an area of the physical model representing a patient tissue which is not a surface of interest; and
a moldable substance for contacting at least a portion of the surface of interest and for maintaining an impression of the surface of interest thereupon;
wherein the moldable substance is solidified into a patient-specific surgical aid, the patient-specific surgical aid being removed from the physical model for use.

14. The system of claim 13, wherein the constraining wall is an outer wall and defines at least one perimeter boundary of the patient-specific surgical aid.

15. The system of claim 13, wherein the constraining wall is an inner wall and defines at least one inner recess of the patient-specific surgical aid.

16. The system of claim 15, wherein the constraining wall is a guide bushing and defines a guiding aperture through a thickness of the patient-specific surgical aid.

17. The system of claim 13, including at least one non-native structure associated with the surface of interest; wherein a molded feature based upon the non-native structure is incorporated in the patient-specific surgical aid.

18. The system of claim 17, wherein the non-native structure is an information feature providing clinically useful information to a user.

19. The system of claim 17, wherein at least a portion of the constraining wall is interposed between at least a portion of the non-native structure and the moldable substance.

20. The system of claim 13, wherein the constraining wall is incorporated into the patient-specific surgical aid.

21. The system of claim 13, wherein the moldable substance is placed into contact with at least a portion of the constraining wall.

22. The system of claim 13, including a intermediate substance which is placed on at least a portion of the surface of interest before the moldable substance is placed into contact with at least a portion of the surface of interest.

23. The system of claim 13, wherein the physical model is based upon a virtual model of the native patient tissue.
